(19) United States
(12) Patent Application Publication
(10) Pub. No.: US 2020/0102563 A1
(43) Pub. Date: Apr. 2, 2020

(54) EXOSOME LOADED THERAPEUTICS FOR TREATING SICKLE CELL DISEASE

(71) Applicant: Exosome Therapeutics, Inc., San Diego, CA (US)

(72) Inventors: Gerardo Rodriguez-Araujo, San Diego, CA (US); Stephen R. Puckett, Sr., Charlotte, NC (US); Stephen R. Puckett, Jr., Charolotte, NC (US); Mitchell W. Puckett, Charolotte, NC (US)

(73) Assignee: Exosome Therapeutics, Inc., San Diego, CA (US)

(21) Appl. No.: 16/591,502

(22) Filed: Oct. 2, 2019

Related U.S. Application Data
(60) Provisional application No. 62/740,396, filed on Oct. 2, 2018.
(60) Provisional application No. 62/769,123, filed on Nov. 19, 2018.

(51) Int. Cl. C12N 15/113; C12N 15/11; C12N 9/22; C12N 15/86

(52) U.S. Cl. CPC C12N 15/113; C12N 15/11; C12N 9/22; C12N 2800/80; C12N 2310/20; C12N 2310/14; C12N 15/86

(57) ABSTRACT

A composition for delivering cargo to cytoplasm of a cell, wherein the cargo treats sickle cell disease. In one embodiment, the composition comprises; an exosome; and cargo, located within the exosome, wherein the cargo comprises short interference RNA (siRNA) with altered single-nucleotide polymorphism SNP rs334 (A) for expressing normal hemoglobin. In another embodiment, the composition comprises: an exosome; and cargo, located within the exosome, wherein the cargo comprises a viral vector containing at least one sequence encoding normal hemoglobin comprising altered SNP rs334.

### CROSS-REFERENCE TO RELATED APPLICATIONS

The present application claims priority to U.S. Provisional Patent Application No. 62/740,396 filed Oct. 2, 2018, and U.S. Provisional Patent Application No. 62/769,123 filed Nov. 19, 2018, the entire disclosures of which are incorporated by reference herein.

### BACKGROUND OF THE INVENTION

### 1. Field of Invention

This invention relates to extracellular vesicles for therapeutic delivery, and more specifically, to compositions and methods of producing exosomes comprising therapeutics to correct mutation in the single nucleotide polymorphism (SNP) rs334 in the chromosome 11, and treat sickle cell diseases in humans and in animals.

### 2. Description of Related Art

Sickle cell disease (SCD) encompasses a group of hematologic disorders caused by a single nucleotide-single gene mutation transposition from a normal adenine to thymine in one or both alleles in the chromosome 11 in the SNP rs334. The transposition of thymine instead of adenine causes the transcription of an abnormal hemoglobin (also called ‘S’ hemoglobin ‘Hb’) that causes intermittent or permanent episodes of ischemia and/or infarction. Sickle hemoglobin changes the anatomy and elastic properties of normal hemoglobin and make red blood cells contained in sickled hemoglobin more viscous with less capacity to transport and deliver oxygen and nutrients to distal organs and tissues. Sickle cells have a ‘C’ shape with earlier cell mortality in comparison with normal red blood cells. Sickle cells accumulate easily in small vessels causing or precipitating coagulation or thrombosis resulting in vessel occlusions or sub-occlusions precipitating pain attacks, strokes, infarctions, etc. This highly impacts the life expectancy and quality of life of SCD patients. In severe forms of SCD, both alleles are affected with a translocation of thymine (T) instead of adenine (A) in SNP rs334. Homozygous sickled hemoglobin, the most severe form, is commonly known as sickle cell anemia in which both alleles are affected (T/T). Heterozygous sickled hemoglobin, a milder form of SCD, affects only one allele (A/T) and patients may or may not manifest signs or symptoms including significant pain and disability due to strokes and infarctions.

Gene therapy has proven to be efficacious in the clinic as supported by FDA-approved drugs for restituting proteins associated with specific diseases. A limitation of gene therapies using a virus include deep genome off target integration issues with the potential of inducing teratogenesis and have the potential to infect other individuals that are in contact with the patient. Specifically, voretigene neparvovec uses adeno-associated virus (AAV) 2, which poses low transduction rates and exposure safety risks due to the development of AAV antibodies. Further, the density of viral copies present in body fluids can pose infection risks to other persons in close contact with a patient (Feria, R. et al. Non-clinical Safety and Efficacy of an AAV2/8 Vector Administered Intravenously for Treatment of Mucopolysaccharidosis Type VI. Mol Ther Methods Clin Dev 6, 143-158 (2017)). The current standard in drug development for SCD involves bone marrow ablation and use of ex vivo lentiviruses. To that end, treatments include hemotransfusions, human leukocyte antigen (HLA)-compatible bone marrow transplantation, and increasing fetal hemoglobin using lentivirus and chemotherapy to ablate the native bone marrow. Bone marrow ablation has undesired effects on pediatric bone and brain development (Bath, L. F. et al. Bone Turnover and Growth during and after Chemotherapy in Children with Solid Tumors. Pediatr Res 55, 224-230 (2004); Iyer, N. S. et al. Chemotherapy-only treatment effects on long-term neurocognitive functioning in childhood ALL survivors: a review and meta-analysis. Blood 126, 346-353 (2015)). Alternatively, drugs (e.g., ticagrelor) to decrease the viscosity of the red blood cells or to inhibit platelet clogging of the arteries are being tested in clinical trials. Such therapeutics aim to target SCD symptoms rather than the SCD genetic defect. Further, current treatment approaches raise ethical, logistical, and scientific concerns about treatment viability.

Currently, researchers are testing viral vectors as a drug delivery system. A primary limitation of using viral vectors in gene therapy is that such lentiviruses are known to integrate into a host cell genome and subsequently create permanent changes in the host cell genome. The resulting effects can be irreversible, for example, in engrafted tissues in humans or chromosomal transpositions. Using retrovirus or lentivirus as a vector in drug delivery has the advantages of transducing hematopoietic stem cells up to 40 percent (%) with high fidelity. This approach, however, is disadvantaged by transgenes (less than 7 Kilo bases (Kb)), difficult in vivo delivery due to low titers, and viral DNA integration in host DNA. Present drug developers face challenges in using viral vectors to deliver target genetic materials to specific cells and specific tissues due to the side effects of viral transfection. Both viral and non-viral vectors have shown modest to poor results and with the caveat of inducing immune responses. Using adeno-associated virus (AAV) as a vector in drug delivery has the advantages of efficient gene transfer 30 to 60%, enabling in vivo administration, relatively safe dosages. This approach is, however, limited by small transgenes (4.7 Kb), inconsistently stable transfer and expression, tissue tropism, and frequently immunogenic. Existing viral and non-viral vectors generate antibodies, anti-viruses or anti-carriers that limit the bioavailability and increase the safety profile of a potential therapeutic product (Arrighetti et al. Exosome-like nanovectors for drug delivery in cancer. Curr Med Chem (2018); Khan et al. Challenges and innovations of drug delivery in older age. Adv Drug Delivery Rev (2018)).

Extracellular vesicles called exosomes are virus-free, bacteria-free, endogenous particles found in all body fluids and body compartments that are highly effective and efficient in cell communication (Arrighetti et al. Exosome-like nanovectors for drug delivery in cancer. Curr Med Chem (2018)). In particular, exosomes exist in body fluids such as blood, urine, and biological secretions. The function of exosomes is to share information such as genetic material (e.g., DNA and RNA), proteins, particles, signals, etc. between cells in a rapid and efficient manner. This biological cell-to-cell communication allows specific cellular microenvironments to synchronize their function and their architecture in response to any stimulus. Exosomes are relatively small and flexible particles of 30-130 nanometers in diameter and composed of the similar materials of normal endogenous cell membranes. Hence, exosomes are highly effective and well-tolerated with minimal to no adverse effects, as a natural cell communication pathway for cells to share information among cells. Genetic material can be inserted into an exosome to be delivered to nearby or distant cells. Exosomes have the advantages of cell transduction up to 100% with high efficiency and fidelity, non-viral and non-immunogenic effects, enabling long transgenes, RNA, proteins, etc. Presently, exosome-mediated drug delivery is challenged by not readily available current good manufacturing practices (cGMP) facilities to produce clinical-grade exosomes, and such exosomes comprise little to no volume of large proteins or antibodies readily available for use in treating human diseases. Further, exosomes can be used as a safe, natural carrier of diagnostics or therapeutic agents in humans (Wei et al. Exosomal miR-1246 in body fluids is a potential biomarker for gastrointestinal cancer. Biomark Med 2018; Chiriaco et al. Lab-on-Chip for Exosomes and Microvesicles Detection and Characterization. Sensors (Basel) 18 (10) (2018)).

In light of these challenges in the field, there exists an unmet medical need for a non-viral drug delivery system to treat the SCD genetic defect rather than remedy only the SCD symptoms. There is a need to produce safe, non-toxic, clinical-grade exosome-mediated therapeutics that pose no immunogenic effects nor detrimental genomic changes. In particular, such an approach would alleviate the required use of chemotherapy or bone marrow ablation, and does not include the use of potentially teratogenic lentiviruses or retroviruses.

### SUMMARY OF THE INVENTION

The present invention overcomes these and other deficiencies of the prior art by providing cGMP autologous and/or universal donor exosomes loaded with cGMP grade genetic materials to correct the SNP causing transcription of abnormal hemoglobin in SCD and associated complications. The present invention provides compositions and methods of producing exosome-mediated therapeutic cargo and delivering the loaded exosome directly into the cytoplasm of cells in tissues for the treatment of diseases including SCD.

The present invention uses exosome-mediated compositions for treating SCD and sickle cell anemia, primary and/or secondary prevention of SCD complications, pain-ischemic attacks, stroke prevention, amputation prevention, SCD pharmacotherapy, mitochondrial dysfunction, oxidative stress, etc. In one embodiment, the present invention provides exosomes loaded with base editors to correct the base mutation in SCD in vitro using CD34+ cells carrying the rs334 T/T.

The present invention provides a highly efficient, ethical, and simple approach to target and correct the genetic defect rather than remedy only the SCD symptoms. Exosomes are highly effective virus-free particles that are well tolerated with minimal to no adverse effects, as they constitute a natural communication pathway for the cells to share information among themselves. An advantage of the present invention is that it poses no concern about human leukocyte antigen (HLA) or major histocompatibility complex (MEW) incompatibility as the present exosomes exist in nature and come from a universal donor, such as the universal donor property of Exosome Therapeutics, Inc.

The advantages of the present invention include restoring the quality of red blood cells to improve sickle cell symptoms and prevent its ischemic cardiovascular complications. In an embodiment, exosome containing miR-126 from CD34+ cells prevent lower extremity loss.

The foregoing, and other features and advantages of the invention, will be apparent from the following, more particular description of the preferred embodiments of the invention and the accompanying drawings.

### BRIEF DESCRIPTION OF THE DRAWINGS

For a more complete understanding of the present invention, the objects and advantages thereof, reference is now made to the ensuing descriptions taken in connection with the accompanying drawings briefly described as follows.

FIG. 1 illustrates a method for producing autologous exosomes from a body fluid according to an embodiment of the invention.

FIG. 2 illustrates a method for producing autologous exosomes according to another embodiment of the invention.

FIG. 3 illustrates a method for producing allogenic exosomes from a cell culture according to an embodiment of the invention.

FIG. 4 illustrates a method for producing allogenic exosomes from a body fluid according to another embodiment of the invention.

FIG. 5 illustrates a table of parameters for exosome isolation and purification according to multiple embodiments of the invention.

FIG. 6 illustrates a schematic of short interference RNA (siRNA) alone and siRNA used in combination with a N-Acetylgalactosamine (GalNAc) construct to deplete expression of mutant hemoglobin according to multiple embodiments of the invention.

FIG. 7 illustrates an exosome meeting cGMP standards comprising a cargo comprising a doxycycline-inducible plasmid DNA to overexpress wild type hemoglobin according to an embodiment of the invention.

FIG. 8 illustrates a cGMP grade-exosome comprising a cargo comprising siRNA and a DNA plasmid construct according to an embodiment of the invention.

FIG. 9 illustrates a schematic of gene editing to correct thymine (T) to adenine (A) in SNP rs334 using clustered regularly interspaced short palindromic repeats (CRISPR)/Cas9, a Zinc finger, a single base editor, or a combination thereof according to various embodiments of the invention.

FIG. 10 illustrates a cGMP grade-exosome comprising a cargo comprising a nuclease base editor according to an embodiment of the invention.

FIG. 11 illustrates insertional mutagenesis to insert a correct rs334 (A) construct into an affected allele of double-stranded DNA (dsDNA) according to an embodiment of the invention.

FIG. 12 illustrates a long terminal repeat (LTR), lentivirus, or retrovirus vector used to express normal hemoglobin according to an embodiment of the invention.

FIG. 13 illustrates a cGMP grade-exosome comprising a cargo comprising a retrovirus used to express normal hemoglobin according to an embodiment of the invention.

FIG. 14 illustrates loading a cGMP grade-exosome comprising a cargo comprising an AAV used to express normal hemoglobin according to an embodiment of the invention.

FIG. 15 illustrates a pharmacokinetic profile after in vivo administration of exosome-mediated cargo according to an embodiment of the invention.

### DETAILED DESCRIPTION OF PREFERRED EMBODIMENTS

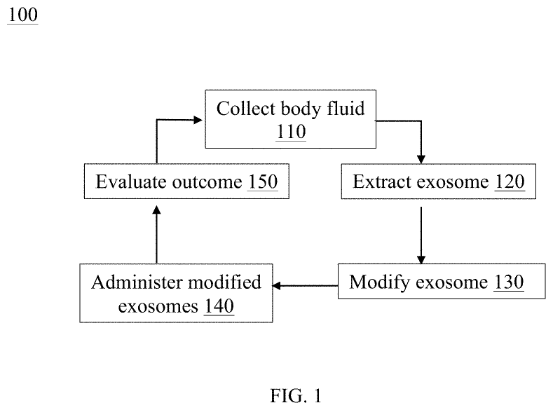

Preferred embodiments of the present invention and their advantages may be understood by referring to FIGS. 1-15, wherein like reference numerals refer to like elements.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Moreover, the described features, structures, or characteristics of the invention may be combined in any suitable manner in one or more embodiments. It will be apparent to those skilled in the art that various modifications and variations can be made to the present invention without departing from the spirit and scope of the invention. Thus, it is intended that the present invention cover the modifications and variations of this invention provided they come within the scope of the appended claims and their equivalents. Reference will now be made in detail to the preferred embodiments of the invention.

The term “exosome” as used herein refers to any extracellular vesicle derived from any body fluid from a human or an animal (e.g., blood), any extracellular vesicle derived from human or animal cell lines, cell cultures, and primary cultures not limited to autologous exosomes, universal donor exosomes, allogenic exosomes, and modified exosomes. In certain examples, the exosome is made to meet pharmaceutical and cGMP.

The term “cargo” as used herein refers to any type of molecule or any type of RNA (microRNA, mRNA, tRNA, rRNA, siRNA, iRNA, regulating RNA, gRNA, long interference RNA, non-coding and coding RNA); any type of DNA (DNA fragments, DNA plasmids, iDNA); including any type of nucleic acid including antisense oligonucleotides (ASO); any genetic material; any genetic construct; any nucleic acid construct; any plasmid or vector; any protein including recombinant endogenous protein, enzyme, antibody, wnt signaling proteins; any lipid; any therapeutic molecule or diagnostic molecule; any cellular component; chimeric antigen receptor T cell (CAR-T cell) transduced without using a retroviruses; any virus including retrovirus, adenoviruses (AdV), AAV of any variety and strain, and DNA viruses; any gene editing technology including CRISPR, CRISPR/Cas9 system, any endonucleases for base editing, a Zinc finger, a single base editor, TALENs, any meganuclease; any synthetic molecular conjugate; or combination thereof loaded into an exosome. Typically, such cargo is naturally not present in the exosome. In certain examples, the cargo is made to meet pharmaceutical and cGMP standards.

In one embodiment cargo could include a promoter. The term “promoter” as used herein refers to any DNA sequence that promotes the transcription of a gene. A plasmid comprises a tissue-specific promoter. Morever, the promoter comprises any tissue-specific promoter (e.g., lung, liver, or any other tissue type), a self-inactivating (SIN) sequence, vesicular stomatitis virus-G protein (VSV-G), or a combination thereof. The advantage of using a tissue-specific promoter is to better target a desired tissue in which to transcribe RNA and subsequently encode a protein.

The term “fluid” as used herein refers to any type of body fluid produced by a human or an animal including but not limited to blood, cerebral spinal fluid, urine, saliva, and any biological secretions, etc.

FIGS. 1-4 illustrate methods of producing exosomes and cargo and methods for exosome loading. Such improved methods and techniques would be appreciated by one of ordinary skill, especially those for increasing yield of purified exosomes and efficient loading of exosome cargo for use in preclinical and clinical studies. The methods of loading the genetic material (e.g., constructs of DNA or RNA, or any type of nucleic acids) directly into exosomes are transformation, transfection and microinjection. In one embodiment, exosomes are extracted, isolated and purified from peripheral blood mononuclear cells (PBMC) circulating in peripheral blood. In such an embodiment, PBMCs are harvested from a patient or a universal donor. PBMCs are isolated and expanded in vitro using closed systems for cell culture. In another embodiment, open systems may be used depending on available resources. PBMCs produce and secrete exosomes into the media of a cell culture. The media can be filtered and exosomes can be sorted by specific parameters and purified to improve exosome quality.

The present extracellular vesicular compositions may be used to treat any of the following diseases including, but not limited to: 1. Cancer and oncological disorders including carcinogenesis, malignancies, tumors, metastasis, nodules of any variety (endodermal, mesodermal or ectodermal origin and due to spontaneous mutations or human papillomavirus or other viral infections); 2. Infectious diseases including human immunodeficiency virus and Ebola viral infections; 3. Cardiovascular disease including coronary arterial disease, peripheral vascular disease, peripheral arterial disease, chronic heart failure (ischemic and non-ischemic), stroke, acute kidney failure, endothelial dysfunction, mitochondrial dysfunction, oxidative stress, etc.; 4. Diabetes mellitus including Type-1 diabetes mellitus and Type-2 diabetes mellitus and any of related complications such as diabetic foot, diabetic retinopathy, peripheral diabetic neuropathy, diabetic kidney disease, insulin resistance, pre-diabetes, gestational diabetes, etc.; 5. Non-alcoholic liver disease, non-alcoholic steatohepatitis, non-alcoholic cirrhosis for primary and secondary prevention; 6. Obesity, overweightness, obesity type-1, type-2, and type 3, morbid obesity, and bariatric surgery; 7. Rare diseases; 8. Gastro-intestinal diseases including Ulcerative colitis, Crohn's disease, etc.; 9. Musculo-skeletal diseases; 10. Sickle Cell Disease. Further, the present extracellular vesicular compositions may be used for cell therapeutics, vector and cell engineering, pharmacology and toxicology assay development, and similar such processes.

The invention provides a gene therapy treatment of SCD using exosomes to deliver a genetic construct to express normal adult hemoglobin and thereby replace sickled hemoglobin with normal hemoglobin in a subject with SCD. This drug delivery can transduce human bone marrow cells to increase the pool of normal adult hemoglobin. As a result, corrected red blood cells shape and function would improve sickle cell symptoms and prevent SCD ischemic cardiovascular complications. In one instance, a composition includes an exosome comprising a cargo comprising a plasmid DNA with a construct to express normal adult hemoglobin.

FIG. 1 illustrates a method for producing autologous exosomes from a body fluid according to an embodiment of the invention. Although the method 100 is illustrated and described as a sequence of steps, its contemplated that various embodiments of the method 100 may be performed in any order or combination and need not include all of the illustrated steps. The method 100 comprises the step of: collecting body fluid 110 from a subject, extracting exosomes 120 from the body fluid, modifying said exosomes 130, administering modified exosomes 140, and evaluating a health-related outcome 150. A health-related outcome in the case of SCD refers to quantifying the delivery of exosomes associated with a cargo for expressing normal hemoglobin in a subject.

In step 110, body fluid is collected from a subject. The subject may be a human or an animal. The body fluid can be peripheral blood, cerebral spinal fluid, secretions, or any other body fluid in which exosomes can be extracted.

In step 120, exosomes are extracted from the body fluid. The extraction method depends on a number of factors including the type of body fluid extracted. Peripheral blood, for example, contains PBMCs and cellular component layers that can be separated by centrifugation at a medical facility. During the extraction process plasma, cells and cellular components are kept on dry ice at all times before isolation.

In one embodiment, the body fluid is transported to a laboratory to undergo isolation. In one embodiment, exosome isolation is achieved using a gradient method or a designated isolation kit (i.e., Total Exosome Isolation kit, ThermoFisher). The isolation kit protocol is highly efficient in yielding high amounts of exosomes from a body fluid, a cell culture media, or cell.

The method 100, provides several approaches to further optimize isolation of exosomes and increase exosome yield from a body fluid. In one embodiment, a gradient column separates components of the collected peripheral blood by cell densities. Such cellular densities correspond to exosomes and exosome-related materials. Another embodiment uses an exosome sorting method, where sorting markers or sorting beads are used to isolate exosomes from solution. A further embodiment uses flow cytometry sorting, which uses surface biomarkers present on exosome to identify and sort exosomes and exosome-related materials from cells and cell suspensions. In one embodiment, an exosome can be modified to include a targeting agent on a surface of the exosome.

Specifically, the exosomes can be modified (modified exosomes) to have specific targeting agents, such as protein epitopes and similar such targeting agents. In various examples, the modified exosome may have a targeting agent covering an entire surface or a partial surface of the extracellular vesicle. Thin layer chromatography can be used to optimally separate exosomes and exosome-related products according to specific exosome associated surface proteins and lipids. An exosome from peripheral blood, for example, would have exosome-related products such as transferrin receptors (immature exosomes), signaling molecules, and similar cellular components. In another embodiment, ionic separation by drift time can be used to optimize isolating exosomes. For example, mass spectrometry may be used to isolate high yields of exosome and exosome-related products. Ion mobility spectrometry-mass spectrometry may also be performed when physicochemical properties of both the exosome and the cargo need to be defined prior to loading into the exosome. Isolated exosome samples can be purified using column methods in accordance with cGMP protocols and regulatory requirements.

In step 130, the exosomes are modified by incorporating cargos. In one embodiment, the modifications to the exosomes are done ex vivo. The exosomes can be further modified to have specific protein epitopes on its surface. Exosomes are assembled or transfected with cargo using a number of methods. In on embodiment depending on the physicochemical properties of the cargo, the exosomes are assembled or transfected with cargo using liposomes (Lipofectamine 2000, Exofect, or heat shock). In another embodiment, exosomes are assembled or transfected with cargo using CAR-T cells transduced without using retroviruses. In another embodiment, exosomes are assembled or transfected with cargo using retroviruses, AdV, AAV of any variety and strain. In another embodiment, exosomes are assembled or transfected with cargo using DNA viruses, siRNA, long interference RNA, noncoding RNA, iRNA, RNA vectors. In another embodiment, exosomes are assembled or transfected with cargo using DNA, DNA plasmids, CRISPR, CRISPR/Cas9 and/or any endonucleases for gene editing. In another embodiment, exosomes are assembled or transfected with cargo using gene editing technology, small molecules, antibodies, and proteins including recombinant endogenous proteins. In another embodiment, exosomes are assembled or transfected with cargo using oligonucleotide therapeutics, including ASO, gene targeting technology, and gene correction technology. In another embodiment, exosomes are assembled or transfected with cargo using synthetic/molecular conjugates and physical methods for delivery of gene and cell therapeutics.

In step 130, the method for loading exosomes efficiently and effectively incorporates autologous or exogenous materials (therapeutic compounds above or any endogenous enzyme, protein, lipid, molecule, DNA or RNA of interest). In non-limiting examples, the method for loading an exosome can include the process of: 1) Lipid-lipid affinity, using liposomes of high and low density; 2) Incorporating intracellular affinity proteins and/or molecules into the exosome; 3) Using clathrin coated vesicles in clathrin-mediated endocytosis methods for incorporation of a therapeutic molecule into an exosome or an exosome-like carrier; and 4) Endocytosis receptors/proteins methodology. In one embodiment the method for loading exosomes includes the methods of exosome membrane dissociation and reconstitution via chemical or electromagnetic gradient changes. In one embodiment, a method is used for large molecules or heavy compounds. In certain examples, the optimization of the method 100 is due to including transmembrane transporters activators when loading the biological materials into the exosomes. After the exosome has been loaded, any potential activator remaining in the exosome will be filtered and purified using column methods in compliance with cGMP and regulatory requirements before undergoing the next processing steps.

Exosomes loaded with cargo are considered mature exosome and are inspected for cGMP compliance, purity and stability for quality assurance and quality check. Next, mature exosomes that have passed the quality check undergo an expansion process if needed. Next, the mature exosomes are diluted and premix into saline/vehicle (depending on the characteristics of the cargo) for a ready to administer tube/cartridge. Finally, the suspension is frozen and stored or shipped to a site for use in clinical or preclinical studies and to patients for self-injection of approved-clinical grade mature exosomes.

In step 140, the mature exosomes are administered to a subject. The subject, may be the same subject from which the body fluid was collected in step 110. The method of administering the exosomes 140 includes, but is not limited to: intravenous, intra-arterial, intra-thecal, intra-ventricular, subcutaneous, subdermal, oral, rectal, intra-peritoneal, transdermal, intraosseous injection, intraosseous infusion, or a combination thereof. In one embodiment the mature exosomes are administered in vivo.

In step 150, the outcome of the treatment is evaluated. This evaluation can be done using a variety of methods, which is immediately apparent to one of ordinary skill in the art.

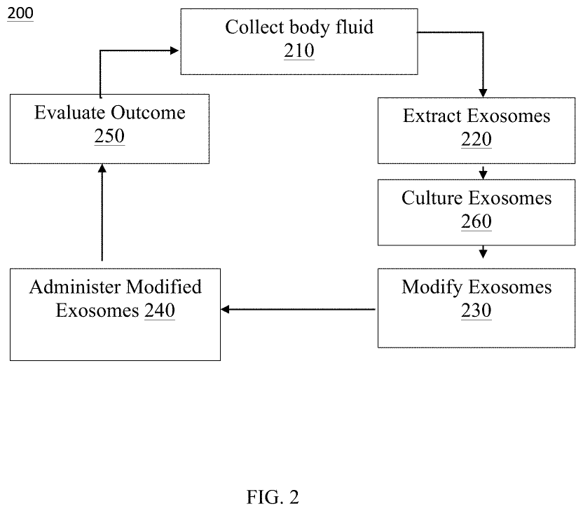

FIG. 2 illustrates a method for producing autologous exosomes from a body fluid according to an embodiment of the invention. Although the method 200 is illustrated and described as a sequence of steps, its contemplated that various embodiments of the method 200 may be performed in any order or combination and need not include all of the illustrated steps. The method 200 comprises the step of: collecting body fluid 210 from a subject, extracting exosomes 220 from the body fluid, culture the exosomes 260, modifying the exosomes 230, administering modified exosome 240, and evaluating the outcome 250.

In step 210, body fluid is collected from a subject. The subject may be a human or an animal. The body fluid can be peripheral blood, cerebral spinal fluid, secretions, or any other body fluid in which exosomes can be extracted.

In step 220, exosomes are extracted from the body fluid using methods as explained above.

In step 260 the exosomes are subjected to a primary culture and expansion. The exosomes will be extracted from primary cultured cells using a gradient or filtration method or a designated expansion kit (i.e., Total Exosome Isolation kit (from cell culture media), ThermoFisher). The cell culture and expansion may be frozen and stored for future exosome isolation procedures/protocols per the methods described above.

In step 230, the exosomes are modified by incorporating cargos. Exosomes are assembled or transfected with cargo using a number of methods as explained above. In one embodiment the step of modifying the exosomes occurs ex vivo.

In step 240 the mature exosomes are administered to a subject using methods as explained above. The step of administering the modified exosomes can occur in vivo or in vitro.

In step 250, the outcome of the treatment is evaluated. This evaluation can be done using a variety of methods, which is immediately apparent to one of ordinary skill in the art.

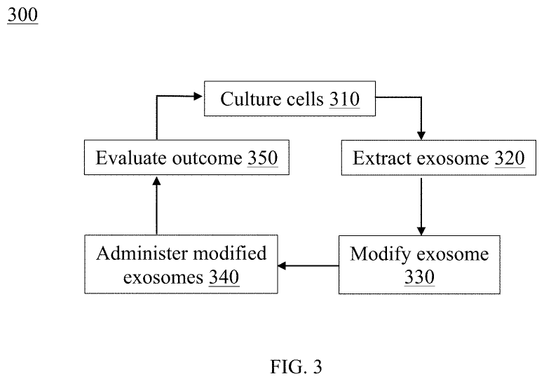

FIG. 3 illustrates a method for producing autologous exosomes from a cell culture according to an embodiment of the invention. Although the method 300 is illustrated and described as a sequence of steps, its contemplated that various embodiments of the method 300 may be performed in any order or combination and need not include all of the illustrated steps. The method 300 comprises the step of: culturing cells 310, extracting exosomes 320 from the cell culture, modifying the exosomes 330, administering modified exosome 340, and evaluating the outcome 350.

In step 310, primary or stable cell lines of human or animal origin are cultured and expanded with standard conditions.

In step 320, exosomes are extracted from the cultured cells.

In step 330, the exosomes are modified by incorporating cargos. Exosomes are assembled or transfected with cargo using a number of methods as explained above.

In step 340 the mature exosomes are administered to a subject using methods as explained above.

In step 350, the outcome of the treatment is evaluated. This evaluation can be done using a variety of methods, which is immediately apparent to one of ordinary skill in the art.

In one embodiment, modifying exosomes, step 330, includes purifying and transducing exosomes in vitro. In such an embodiment, the exosome undergoes in vitro testing of transduction properties such as using human bone marrow cells and primary culture of PBMCs. In an embodiment, to form an exosome composition, an exosome undergoes transduction by a DNA plasmid construct that expresses normal hemoglobin. The loaded exosomes and therapeutic cargo are tested for stability. In such an embodiment, during step 350, the exosome compositions are delivered in vitro. Further, the loaded exosomes can be tested with human bone marrow cells and primary culture of sickle cell bone marrow.

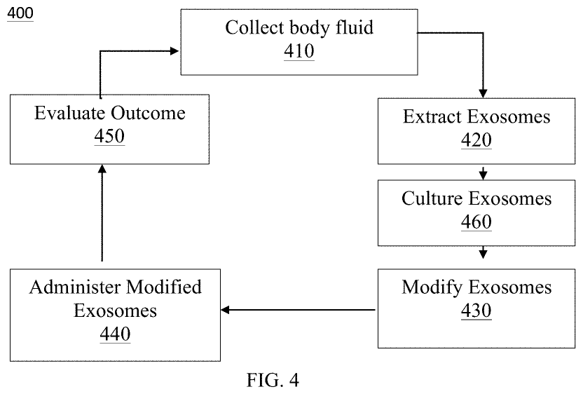

FIG. 4 illustrates a method for producing autologous exosomes from body fluid according to an embodiment of the invention. Although the method 400 is illustrated and described as a sequence of steps, its contemplated that various embodiments of the method 400 may be performed in any order or combination and need not include all of the illustrated steps. The method 400 comprises the step of: collecting body fluid 410, extracting exosomes 420 from the body fluid, culturing the exosomes 460, modifying the exosomes 430, administering modified exosome 440, and evaluating the outcome 450.

In step 410, a body fluid is collected from a universal donor or patient. The subject may be a human or an animal. The body fluid can be peripheral blood, cerebral spinal fluid, secretions, or any other body fluid in which exosomes can be extracted.

In step 420, exosomes are extracted from the body fluid using methods as explained above.

In step 460, the exosomes are cultured. The exosomes are expanded using a primary cell culture from the body fluid of the universal donor or patient using a gradient method or a designated isolation kit (i.e., Total Exosome Isolation kit, ThermoFisher). The isolation kit protocol is highly efficient in yielding high amounts of exosomes from either body fluids or cell culture media or cell. The cell culture and expansion from the universal donor or patient may be frozen and stored for future exosome isolation procedures/protocols per the methods described above.

In step 430, the exosomes are modified by incorporating cargos. Exosomes are assembled or transfected with cargo using a number of methods as explained above.

In step 440 the mature exosomes are administered to a subject using methods as explained above.

In step 450, the outcome of the treatment is evaluated. This evaluation can be done using a variety of methods, which is immediately apparent to one of ordinary skill in the art.

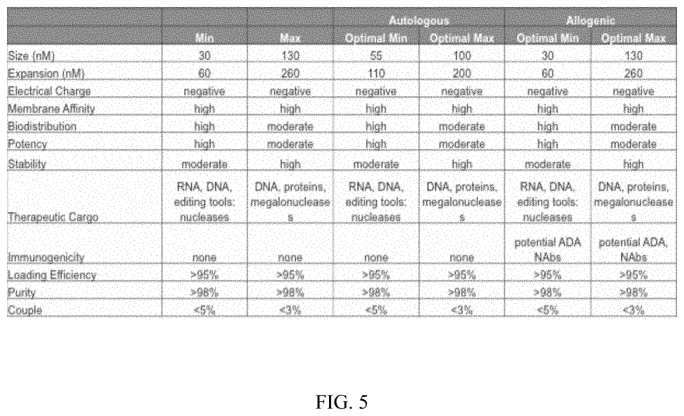

FIG. 5 illustrates the parameters used to sort exosomes according to an embodiment of the invention. In one embodiment, the invention provides autologous exosomes having a vesicle size between about 55 nanometers (nM) and 100 nM. In certain embodiments, allogenic exosomes have a vesicle size between about 30 nM and 130 nM. A vesicle size between 55 nM and 100 nM may be chosen as larger exosomes are less stable. Also, larger exosomes can couple with other exosomes making calculating drug dose, bioavailability, and biodistribution challenging. In some embodiments, the exosomes have the ability to expand to a size between about 60 nM and 260 nM. Such expanded exosomes can encompass large constructs. In some embodiments, the expanded exosomes can encompass more than or equal to about 7 kilo bases (Kb), and accommodate one or more copies of a relatively large viral particle such as an AAV. In one example, an exosome is loaded with at least four AAV particles to improve an exosome safety profile. In some embodiments, either an autologous or allogenic exosome has a negative electrical charge. Both autologous and allogenic exosomes can have a high membrane affinity. In some embodiments, biodistribution may be moderate to high. In a similar embodiment, potency may range from high to moderate while stability may be moderate to high.

In an embodiment, exosomes can comprises a smaller sized cargo comprising RNA, DNA, editing tools (e.g., nucleases), or combination thereof. In another embodiment, an exosome can comprise a larger cargo comprising DNA, proteins, megalonucleases, or a combination thereof. One advantage of autologous exosomes is that they do no illicit a significant immune response. Allogenic exosomes may illicit anti-drug antibodies (ADA) and neutralizing anti-bodies (NAb). One embodiment of the present invention enables high efficiency of loading cargo into at least ninety-five percent (95%) of exosomes. Another embodiment can provide a higher purity of exosomes of at least ninety-eight percent (98%).

Additionally, an exosome can further be modified to include a targeting agent on a surface of the exosome. For example, an exosome can have specific protein epitopes, plasma membrane components, etc.

Human sickled hemoglobin arises from a mutation occurring at SNP rs334 with a translocation of thymine (T) instead of adenine (A) in one or both alleles in chromosome 11. The SCD genetic defect can be corrected by efficiently and effectively delivering exosome-mediated cargo containing genetic constructs that either deplete expression of mutant hemoglobin or express (overexpress) normal hemoglobin containing adenine at SNP rs334 (A) instead of SNP rs334 (T). Sorted and purified exosomes are loaded with a cargo for drug delivery. For clinical use in humans, the exosomes and the cargo both meet pharmaceutical or cGMP standard. Specific methods for optimizing isolation and purification of exosomes, cargo targeting sickle cell diseases, and exosome-mediate cargo delivery are described above.

In one embodiment, during acute care of thrombotic events of SCD, the exosome-mediated cargo can be combined with low frequency ultrasound in stroke or myocardial infraction causing-thrombus to maximize the effect thrombolytic agents (i.e., tissue plasminogen activator, etc.). Exosomes loaded with a cargo are preferably administered after low frequency ultrasound delivery.

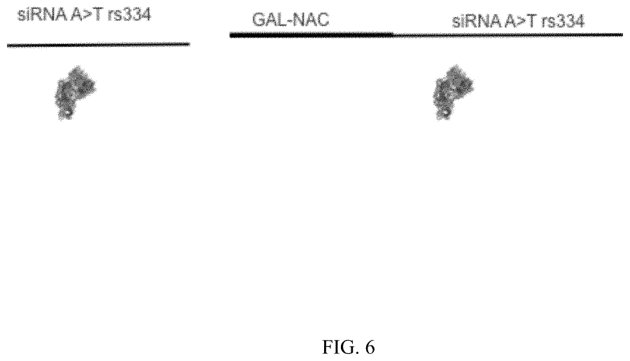

Gene silencing using siRNA or ASO is useful for inhibiting the translation of sickled hemoglobin and for posttranslational silencing of sickle hemoglobin in patients with SCD. FIG. 6 illustrates a siRNA and a siRNA-GalNAc construct used to deplete expression of mutant hemoglobin according to multiple embodiments of the invention. In this example, the siRNA comprises correct SNP rs334 (A) for expressing human normal hemoglobin. In one example, the invention provides an exosome-mediated cargo to be delivered to a subject with SCD. The cargo is loaded into an exosome for drug delivery in vivo or in vitro. In certain instances, a cargo comprises siRNA, a GalNAc construct, or a combination thereof to express normal hemoglobin. After the exosome-mediated cargo reaches the cytoplasm of a cell in a subject with sickle cell disease, the cargo represses the expression of sickled hemoglobin (mutant SNP rs334 (T)). In one embodiment, the siRNA and siRNA-GalNAc constructs are modified to enable greater loading efficacy.

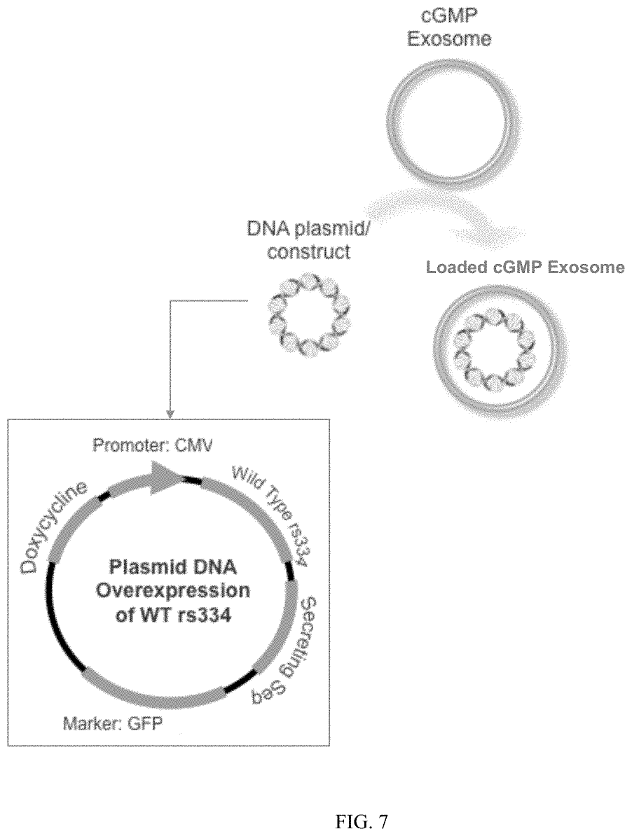

FIG. 7 illustrates an exosome meeting cGMP standard loaded with a plasmid DNA according to an embodiment of the invention. The invention provides a composition for delivering a cargo to the cytoplasm of a cell in a subject with sickle cell disease, the composition comprising: an exosome comprising the cargo, wherein the cargo comprises a DNA plasmid comprising a sequence encoding normal hemoglobin, and the cargo is naturally not present in the exosome. As shown, the plasmid DNA is a doxycycline-inducible plasmid DNA to express or overexpress wild type hemoglobin (normal hemoglobin). In this example, a cargo comprises a DNA plasmid comprising a promoter targeting a specific tissue, wild type rs334 sequence expressing the correct version of rs334 (A), and a secreting sequence. Optionally, the DNA plasmid includes a marker sequence encoding green fluorescent protein (GFP). In multiple examples, the DNA plasmid is induced by ampicillin, kanamycin, or another equivalent agent recognized by one of ordinary skill. In one example, the promoter is cytomegalovirus (CMV). In other examples, the promoter is any tissue-specific targeting promoter. The wild type hemoglobin sequence encodes adult human hemoglobin. In specific examples, a cargo comprises siRNA, siRNA-GalNAc construct, plasmid DNA, another iRNA technology, or a combination thereof. In certain instances, a cargo includes only a DNA plasmid as a monotherapy in preclinical and clinical trials as well for human use. An exosome is modified or transduced ex vivo with a cargo. Exosome delivered human adult hemoglobin expression vector DNA in combination with an siRNA for silencing sickle hemoglobin to treat sickle cell disease.

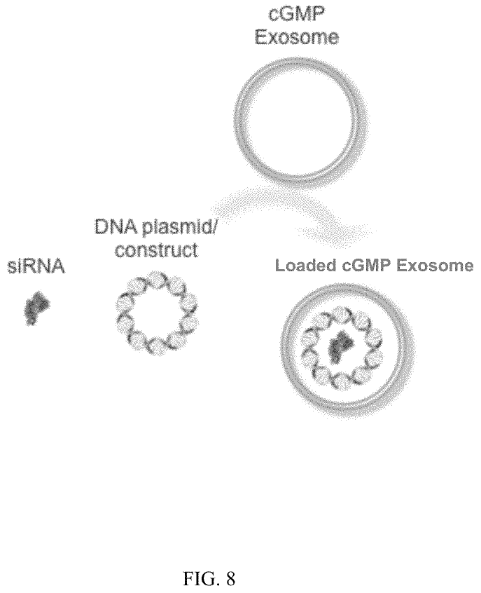

FIG. 8 illustrates a cGMP-grade exosome loaded with a cargo comprising siRNA and a DNA plasmid according to an embodiment of the invention. In an alternative example, a cGMP grade-exosome comprises siRNA or a DNA plasmid. A loaded exosome comprising a plasmid DNA construct to express normal hemoglobin and siRNA against sickle hemoglobin sequences facilitates increasing a pool of normal hemoglobin and reducing the quantity of abnormal sickle hemoglobin in vivo or in vitro. The siRNA binds sickle hemoglobin to inhibit abnormal protein function. Specifically, reducing the amount of sickled hemoglobin in erythroblasts facilitates the prospective formation of normal shaped red blood cells originating from bone marrow. The DNA plasmid expresses normal hemoglobin to restore and/or increase the pool of normal shape and function of red blood cells. Thus, sickle cell disease is treated by reestablishing hemoglobin function and improving its associated complications. The present exosome-mediated cargo significantly improves transduction efficacy and precision of delivery to cellular targets and safety profiles without immunogenicity.

FIGS. 7-8 illustrate exosomes loaded with different types of cargo. In an embodiment, any number of cargos discussed may be loaded into a single exosome. Further, the different types of cargo may be loaded into exosomes in any number of combinations. In one embodiment, the exosome may have two or more cargos wherein the two or more cargos may be identical or substantially the same. In another embodiment, an exosome may have two or more cargos wherein each of the two or more cargos are distinct from one another.

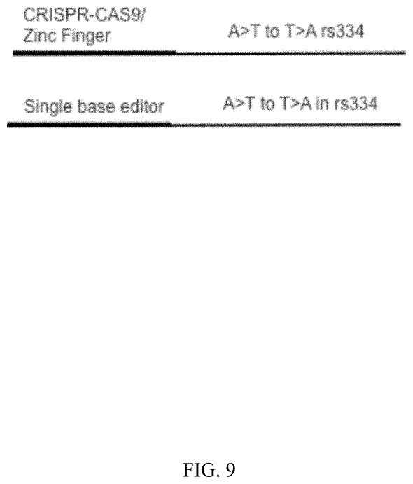

In various examples, a cargo is exposed to gene editing materials to correct the SNP rs334. FIG. 9 illustrates a schematic of gene editing to correct thymine (T) to adenine (A) in the SNP rs334 using CRISPR CAS9, Zinc finger, a single base editor, or a combination thereof according to various embodiments of the invention. For example, Zinc finger domains can be engineered to target a specific desired DNA sequence and this enables a zinc-finger nuclease to target unique sequences within a genome such as SNP rs334 in SCD. By taking advantage of endogenous DNA repair machinery, these reagents can be used to precisely alter the genome by editing the SNP rs334. CRISPR-Cas9 is short for clustered regularly interspaced short palindromic repeats and CRISPR-associated protein 9. CM:SPR is a highly precise gene-editing tool that relies on guide RNA (gRNA) to direct a scissor-like Cas9 enzyme to the desired spot in the genome to correct a misspelling such as thymine (T) to adenine (A) at SNP rs334. Such CRISPR system in combination with other enzymes are used to directly install a point mutation into cellular DNA or RNA without making double-stranded DNA breaks. Typically, the gene editing to modify exosomes and cargo occur ex vivo before the modified exosomes having a cargo are administered to a patient. Alternatively, a cargo comprises a nuclease single base editor, TALENs, a meganuclease, a wnt signaling protein, or a combination thereof.

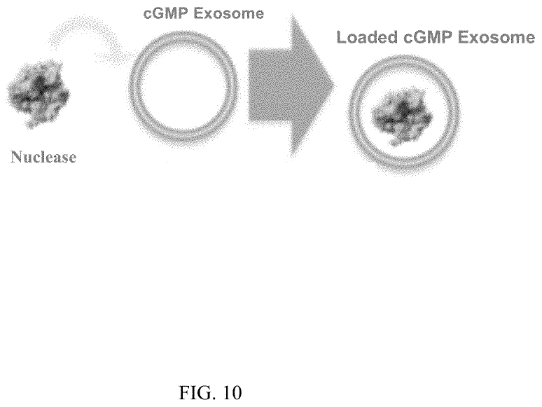

FIG. 10 illustrates a cGMP grade-exosome comprising a cargo comprising a nuclease according to an embodiment of the invention. The nuclease functions as a base editor to correct thymine for adenine in the SNP rs334. In vitro, an exosome mediated-nuclease delivery enables a nuclease to correct the base mutation in CD34+ cells carrying the SNP rs334 (T/T) or SNP rs334 (A/T). In specific examples, a loaded exosome comprising a nuclease base editor is delivered to the affected CD34+ cells. Base editors are the latest generation of gene editing tools with very high precision at targeting single nucleotides within a sequence. Ideally, the loaded exosomes meeting clinical-grade GMP, regulatory chemistry manufacturing and controls (CMC) compliance can be deployed to patients that suffer from SCD and its complications. The invention enables SCD treatment without the use of chemotherapy or bone marrow ablation using teratogenic lentiviruses or retroviruses.

One method of producing normal adult hemoglobin using CD34+ cells expressing the SCD mutation includes the step of: isolating and purifying exosomes, generating a base editor for a specific mutation or sequence, loading the base editor into an exosome, and assessing pharmacokinetics, pharmacodynamics and toxicology in vitro of the exosome-delivered base editor.

Generate nuclease base editor for SCD. Base editors show very low (0.1%) indel formation (insertion or deletion of bases in the genome) which makes it safe for therapeutic use. A nuclease base editor enables treating SCD by targeting and correcting one or both alleles at SNP rs334. First, a single guide RNA (sgRNA) is designed and added to a nuclease base editor plasmid to increase precision on a target DNA sequence (SNP rs334 (T)). Second, a protospacer, protospacer adjacent motif (PAM) sequence, and motifs surrounding SNP rs334 (T) are included in the target DNA sequence. Inclusion of a protospacer and a PAM sequence enable the CRISPR-Cas9 system to cleave the target DNA sequence. Thirdly, the expression plasmid with sgRNA is cloned. Lastly, the sgRNA and the nuclease base editor are loaded into an exosome. A nuclease base editor corrects one or both alleles at SNP rs334.

Loading base editor into exosomes. The proportion of loading is 1:1 (exosome: base editor) using proprietary techniques that include electromagnetism and membrane dissociation technologies. Exosomes meeting the standards mentioned and having a vesicle size between fifty-five (55) and one hundred (100) nM are selected for cargo loading.

Assessing pharmacokinetics, pharmacodynamics and toxicology in vitro. In one preferred example, a loaded exosome comprising a nuclease base editor is added to a media containing CD34+ cells with the SCD defect at SNP rs334 (T). Pharmacokinetic measures include: time to exosome disappearance in the media, Cₘₐₓ, Tₘₐₓ, T1/2, etc. Pharmacodynamics include time course and dose range finding for RNA and protein detection with the corrected hemoglobin and sickle hemoglobin. Genome DNA sequencing was used to confirm the correction of the SCD mutation. Toxicology include cell apoptosis analysis (TUNEL) and cell viability analyses (for assessing cell metabolic activity), genotoxicity (chromosome aberration, gene mutation test, DNA damage and repair, unscheduled DNA synthesis).

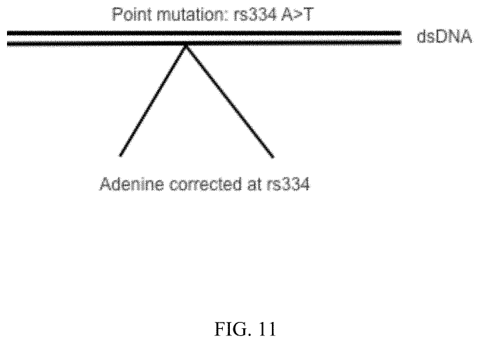

The SCD point mutation occurring at SNP rs334 can be corrected using insertional mutagenesis. FIG. 11 illustrates insertional mutagenesis to insert a correct rs334 (A) construct into an affected allele of double-stranded DNA (dsDNA). Transposing or exchanging the thymine (T) for adenine (A) at rs334 enables the transcription of normal hemoglobin instead of sickled hemoglobin. For example, the genotypes A/T or T/T can be corrected to A/A or A/T at SNP rs334. The constructs produced from insertional mutagenesis include plasmid DNA, transposons, and sleeping beauty transposon systems. A cargo comprising such constructs are safe to deploy directly into animals and humans. In specific examples, the cargo comprising such constructs undergo ex vivo engineering of a specific cell lineage such as hematopoietic stem cells (HSC), other hematic cells, any cell types in the body, cells with carrier, and cells without a carrier. The carrier is one selected from a group consisting of: an exosome, a modified exosome or a viral vector. Examples of a viral vector include a lentivirus, retrovirus, AdV and AAV which can cause permanent or transient integration into a host genome leading to short term or long term effects.

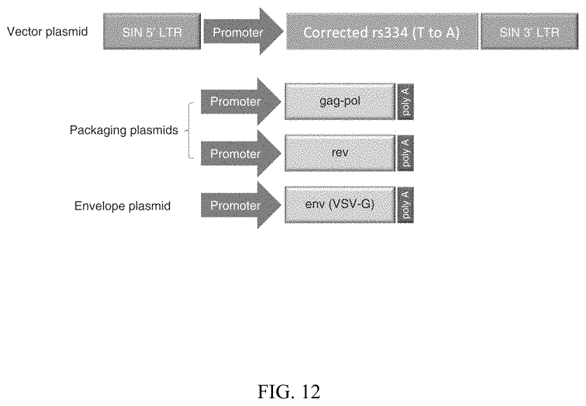

Furthermore, a cargo comprises viral vectors to express normal hemoglobin. FIG. 12 illustrates a schematic of a LTR, lentivirus/retrovirus vector used to express corrected SNP rs334. As shown, the viral vector comprises a tissue-specific promoter, for example, CMV. Moreover, the promoter comprises any tissue-specific promoter (e.g., lung, liver, or any other tissue type), a self-inactivating (SIN) sequence, vesicular stomatitis virus-G protein (VSV-G), or a combination thereof. The advantage of using a tissue-specific promoter is to better target a desired tissue in which to transcribe RNA and subsequently encode protein such as normal hemoglobin.

In several examples, an extracellular vesicle is an autologous exosome, a universal donor exosome, or a combination thereof. In other examples, a modified exosome may comprise specific protein epitopes for improved efficacy of exosome loading or better exosome delivery. Preferably, an exosome meets cGMP standards.

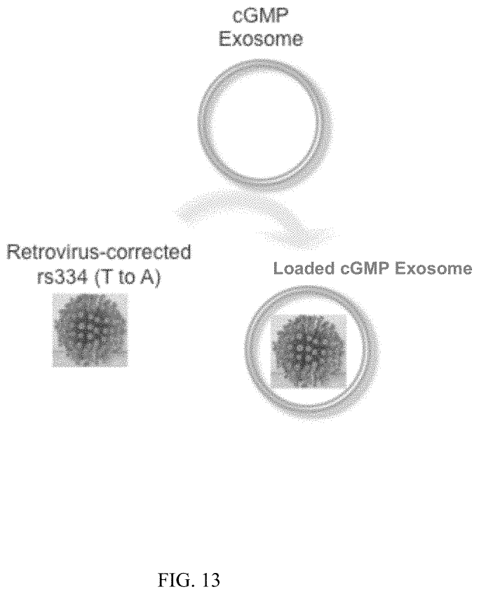

FIG. 13 illustrates a cGMP exosome with a cargo comprising a retrovirus for expressing corrected rs334 according to an embodiment of the invention.

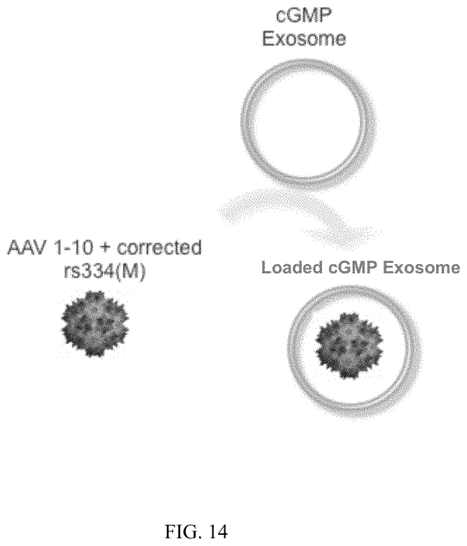

FIG. 14 illustrates a cGMP grade-exosome with a cargo comprising an AAV for expressing corrected rs334 according to an embodiment of the invention.

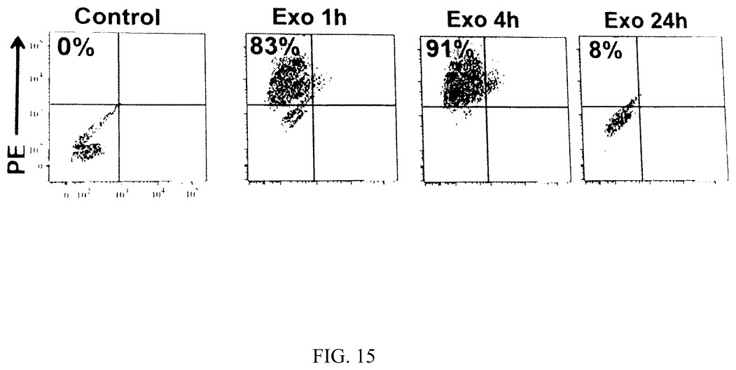

FIG. 15 illustrates a pharmacokinetic profile after in vivo administration of loaded exosomes according to an embodiment of the invention. The exosomes injected intravenously into an animal model of myocardial ischemia showed most exosomes were taken up by tissues in less than 24 hours. Only a smaller quantity of exosomes (8%) continued circulating after 24 hours. These data support how the present exosomes enable improved in vivo delivering of cargo to tissues.

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments and claims.

1. A composition for delivering cargo to cytoplasm of a cell, wherein the cargo treats sickle cell disease, the composition comprising:
an exosome; and
cargo, located within the exosome, wherein the cargo comprises short interference RNA (siRNA) with altered single-nucleotide polymorphism SNP rs334 (A) for expressing normal hemoglobin.

2. The composition of claim 1, wherein the exosome is isolated from autologous cells of a subject or a universal donor.

3. The composition of claim 1, wherein the exosome is isolated from a cell line, a primary cell culture, or a combination thereof.

4. The composition of claim 1, wherein the exosome is isolated from a stem cell.

5. The composition of claim 1, wherein the cargo further comprises an siRNA-N-Acetylgalactosamine (GalNAc) construct.

6. The composition of claim 1, wherein the cargo further comprises a CRISPR-CAS9 system, a Zinc finger, a single base editor, or a combination thereof.

7. The composition of claim 1, wherein the cargo further comprises a viral vector containing at least one sequence encoding normal hemoglobin comprising altered SNP rs334.

8. The composition of claim 1, wherein the exosome comprises at least one targeting agent.

9. The composition of claim 8, wherein the at least one targeting agent is a protein epitope.

10. The composition of claim 1, wherein the cargo further comprises at least one plasmid.

11. The composition of claim 10, wherein the at least one plasmid comprises a promoter, wild type sequence of rs334, and a secreting sequence.

12. A composition for delivering cargo to cytoplasm of a cell, wherein the cargo treats sickle cell disease, the composition comprising:
an exosome; and
cargo, located within the exosome, wherein the cargo comprises a viral vector containing at least one sequence encoding normal hemoglobin comprising altered SNP rs334.

13. The composition of claim 12, wherein the cargo further comprises a CRISPR-Cas9 system, a Zinc finger, a single base editor, or a combination thereof.

14. The composition of claim 12, wherein the cargo further comprises at least one plasmid.

15. The composition of claim 14, wherein the at least one plasmid is a retrovirus or an adeno-associated virus (AAV).

16. The composition of claim 14, wherein the at least one plasmid is a RNA plasmid, a DNA plasmid, or a combination thereof.

17. The composition of claim 14, wherein the at least one plasmid comprises a promoter, wild type sequence of rs334, and a secreting sequence.

18. The composition of claim 17, wherein the promoter is cytomegalovirus (CMV).

19. The composition of claim 14, wherein the at least one plasmid further comprises a marker sequence.

20. The composition of claim 19, wherein the marker sequence encodes green fluorescent protein.
